(19) United States
(12) Patent Application Publication
(10) Pub. No.: US 2005/0260544 A1
(43) Pub. Date: Nov. 24, 2005

(54) Novel device

(76) Inventors: Stephen Jones, Surrey (GB); Graham Jackson, Surrey (GB); Andrew Macleod, Hertfordshire (GB); Michael Noble, Hertfordshire (GB); Timothy Wood, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/487,018

(22) PCT Filed: Aug. 15, 2002
(86) PCT No.: PCT/EP02/09121

§ 371 Date: Feb. 17, 2004

(30) Foreign Application Priority Data
Aug. 17, 2001 (GB) 0120136.7
Aug. 17, 2001 (GB) 0120144.1
Jan. 16, 2002 (GB) 0200871.2

(51) Int. Cl. A61K 7/16; A61C 5/00

(52) U.S. Cl. 433/217.1; 424/49

(57) ABSTRACT

A device for delivering an oral healthcare substance to the teeth, gingival and/or mucosal tissues comprising a strip of an orally acceptable flexible material with an oral healthcare substance deposited onto it or impregnated into its bulk, capable of adhesion to a tooth surface but with the adhesion function being provided independent of the oral healthcare substance. A preferred device comprises a strip of a plastically deformable material, to which is attached a layer of an absorbent material, with a peroxide-containing tooth whitening gel on the layer of absorbent material.

The present invention relates to a device for the delivery of an oral care substance or composition to oral surfaces including teeth, gingival and mucosal tissues.

Delivery devices are known for delivering an oral healthcare substance to the surface of a tooth and oral tissue, comprising a strip of a flexible film on a surface of which is an oral healthcare substance which also acts as an adhesive to attach the device to a tooth surface. See for example WO-A-98/55044, U.S. Pat. No. 5,989,569, U.S. Pat. No. 5,879,691, U.S. Pat. No. 5,894,017, WO-A-98/55079, WO-A-95/16488. U.S. Pat. No. 6,045,811, WO-A-00/07518 and U.S. Pat. No. 2,835,628.

The problem with these devices is that they are difficult to handle and unpleasant in use. From the consumer perspective, primary importance would be a low cost, non-bulky oral care delivery device that is both easy, effective and unobtrusive in use.

It is an object of this invention to provide alternative devices for delivering an oral healthcare substance to the surface of a tooth. Known devices in which the oral healthcare substance also provides an adhesive function suffer from the disadvantage that for example compatible adhesive and oral healthcare substances must be used, and the present invention aims to alleviate this problem.

According to this invention a delivery device for delivering an oral healthcare substance to the oral surfaces of the teeth, gingival and/or mucosal tissues is provided, comprising;

a strip of an orally acceptable flexible material, having a strip surface capable of being applied to a tooth surface and/or adjoining soft tissue,

the strip having an oral healthcare substance deposited upon a strip surface thereof, and/or impregnated into its bulk, the substance being capable of transference from the strip surface to the tooth surface and/or adjoining soft tissue,

the strip being capable of adhesion to a tooth surface and/or adjoining soft tissue to which the strip is applied,

the adhesion function being provided independent of the oral healthcare substance.

By separation of the adhesive function and oral healthcare substance, greater flexibility of construction options are made available.

The strip of an orally acceptable material may be any material, natural or synthetic, which can be applied to the surface of a tooth by a user, adapted by pressure, e.g a pinching action, to the contours of the surface of a tooth, and subsequently easily removed by the user. The strip should be flexible enough to enable such adaptation to the tooth surface.

This adaptation may be via permanent deformation i.e. in which the strip undergoes plastic deformation with little or no tendency to return to its original shape after adaptation to a tooth surface. For example the strip may comprise a plastically deformable material. By plastically deformable it is meant that the material, at least when in the form of the strip, may be easily deformed by the user using finger or hand pressure, below or at body temperature, so as to fit the device to the overall shape of the user's teeth, preferably also being capable of being deformed into the gaps between the user's teeth. Typically a plastically deformable device should be plastically deformable under the application of a pressure of less than 250,000 pascals, e.g. to allow the strip to be deformed to fit the contours of the users teeth and/or oral tissues. Suitable plastically deformable materials include waxes, in particular dental waxes, e.g. of the known type suitable for making casts of the shape of teeth. The plastically deformable materials preferably comprises such a wax. Such a plastically deformable material may provide the adhesive function by means of the material being plastically deformed to closely conform to the shape of the teeth and to the spaces between the teeth, and thereby gripping the teeth independent of any adhesive function provided or not provided by the oral healthcare substance. The gripping action provided by such a plastically deformable material may by by friction between the material and the surfaces of the teeth and/or oral tissues to which it is applied, and/or by the “deadfold” action as the material accomodates to the contours of the surfaces and fits into surface concavities etc. to thereby grip.

Alternatively the adaptation of the strip to the tooth surface may be via non-permanent deformation i.e. in which the strip deforms elastically and returns to or nearly to its original shape after removal from the tooth surface.

The strip material should be compatible with the oral cavity and comfortable for the user. The material may comprise a polymeric film or a fabric material, woven or non-woven. The material may be transparent or opaque, or may be coloured to be visually unobtrusive, or alternatively noticeable when in the mouth, dental waxes as mentioned above being available in a variety of grades and colours such as white, blue and pink. The material may have matter printed thereon, e.g. a manufacturer's or retailer's logo, or other visible symbol. Alternately or additionally the material of the strip may contain a colourant, for example titanium dioxide to impart a bright whiteness to the strip material.

The strip may be single or multiple layered. For example different layers of a multiple layer strip may impart respective advantageous properties. The oral healthcare substance may be applied directly to the strip material, for example the oral healthcare substance may be applied as a layer to a strip of a plastically deformable material such as a wax, and the device may comprise one or more wax layer with such an oral healthcare substance deposited as a layer thereon. Alternately for example there may be an inner (i.e. closer to or adjacent to the tooth surface) carrier layer able to carry the substance, and an outer barrier layer, e.g. to prevent escape or loss of the substance during use. For example one or more layer may be an open or closed cell foam, and an open celled foam may advantageously carry the substance in its cells. For example to at least one surface of the strip there may be attached a carrier layer of an absorbent material. Such an absorbent material is preferably a fabric, woven or non-woven, preferably non-woven. A suitable non-woven fabric is for example, polypropylene, viscose or a polypropylene-viscose blend. The absorbent material may occupy the whole of the said at least one surface of the strip.

For example another type of multiple layered strip may comprise an outer backing layer as mentioned above and an inner, contact layer to be placed adjacent a tooth or other oral tissue surface, the inner layer being porous and the oral healthcare substance being provided between the inner and outer layers to be released through the porous contact layer during use. The outer layer may for example comprise a plastically deformeable wax as described herein, and the inner layer may comprise a similar wax. The outer and inner layers may define a pocket between them for the oral healthcare substance. The inner layer may comprise a porous material such as a foam material, or an absorbent e.g. non-woven fabric, or it may comprise a relatively impermeable material perforated with plural holes. It may be necessary to provide the contact layer of such a strip with a removeable protection layer prior to use. Such a strip could for example be made by depositing the substance in a patch on the outer layer then laminating the inner layer to the outer layer, suitably forming a bond between the layers around the patch.

The strip material may for example be textured, to provide a surface more able to carry the substance or for user comfort etc. For example the strip material may have wells or dimples in its surface, or may have a sponge-like surface, to contain the substance and/or an adhesive material. The strip material may be hydrophilic, and for example may be a hydrogel polymer, of generally known type. Alternatively the strip material may be hydrophobic. For example a multiple layered strip may comprise one or more hydrophilic layer and one or more hydrophobic layer. The strip material, or the material of one or more of the layers of a multiple layer strip, may be biodegradable in the mouth environment. Suitable film materials are for example the cellulose or polyethylene based materials disclosed respectively in U.S. Pat. No. 2,835,628 and WO-A00/07518, hydroxypropylmethylcelluloses, and fluorinated polymers such as PTFE.

The shape and size of the strip may be such that it can be applied to either a single tooth, for example being of a shape and size corresponding to a surface of an individual tooth, or preferably to a plurality of teeth simultaneously, suitably therefore being an elongate strip of a length corresponding generally to the length of a plurality of teeth to which it is to be applied, and a width corresponding generally to the height of the teeth. Alternatively and preferably the width of the strip may be greater than the height of the teeth to enable the strip to be bent or folder over the crown and behind the teeth to contact the rear surfaces of the teeth.

The term “oral healthcare substance” as used herein includes curative, prophylactic and cosmetic active substances or compositions thereof. Examples of the oral conditions these substances may address include, but are not limited to one or more of, appearance and structural changes to teeth, whitening, stain bleaching, stain removal, plaque removal, tartar removal, cavity prevention and treatment, inflamed and/or bleeding gums, mucosal wounds, lesions, ulcers, aphthous ulcers, cold sores, tooth abscesses, tooth and/or gum pain, tooth sensitivity (e.g. to temperature changes), and the elimination of mouth malodour resulting from the conditions above and other causes such as microbial proliferation.

Suitable oral care actives include any substance that is generally considered as safe for use in the oral cavity and that provides a change to the overall health of the oral cavity. The level of oral care substance in the present invention may generally be from about 0.01% to about 40%, preferably from about 0.1% to 20%.

Oral care substances of the present invention may include many of the actives previously disclosed in the art. The following is a non all-inclusive list of oral care actives that may be used in the present invention.

Teeth whitening actives may be included in the oral care substance of the present invention. The actives suitable for whitening are selected from the group consisting of peroxides, metal chlorites, perborates, percarbonates, peroxyacids, and combinations thereof. Suitable peroxide compounds include: hydrogen peroxide, calcium peroxide, carbamide peroxide, and mixtures thereof. A preferred peroxide is hydrogen peroxide. Suitable metal chlorites include calcium chlorite, barium chlorite, magnesium chlorite, lithium chlorite, sodium chlorite and potassium chlorite. Additional whitening actives may be hypochlorite and chlorine dioxide. A preferred chlorite is sodium chlorite.

Anti-tartar agents that are known for use in dental care products includes phosphates. Phosphates include pyrophosphates, polyphosphates, polyphosphonates and mixtures thereof. Pyrophosphates are among the best known for use in dental care products. Pyrophosphate ions delivered to the teeth derive from pyrophosphate salts. The pryophosphate salts useful in the present compositions include the dialkali metal pyrophosphate salts, tetra-alkali metal pyrophosphate salts, and mixtures thereof. Disodium dihydrogen pyrophosphate (Na₂H₂P₂O₇), tetrasodium pyrophosphate (Na₄P₂O₇), and tetrapotassium pyrophosphate (K₄P₂O₇) in their unhydrated as well as hydrated forms are preferred. Anticalculus phosphates include potassium and sodium pyrophosphates; sodium tripolyphosphate; diphosphonates, such as ethane-1-hydroxy-1,1-diphosphonate, 1-azacycloheptane-1,1-diphosphonate, and linear alkyl diphosphonates; linear carboxylic acids and sodium and zinc citrate.

Agents may be used in place of or in combination with the pyrophosphate salt include materials as synthetic anionic polymers including polyacrylates and copolymers of maleic anhydride or acid and methyl vinyl ether (e.g. Gantrez™), as described, for example, in U.S. Pat. No. 54,627,977, to Gaffar et al, as well as e.g. polyamino propane sulfonic acid (AMPS), zinc citrate trihydrate, polyphosphates (e.g. tripolyphosphate; hexametaphosphate), diphosphonates (e.g. EHDP, AHP), polypeptides (such as polyaspartic and polyglutamic acids), and mixtures thereof.

Fluoride ion sources are well known for use in oral care compositions as anticaries agents. Fluoride ions are included in many oral care compositions for this purpose, and similarly may be incorporated in the invention in the same way.

Antimicrobial agents can also be present in the oral care compositions or substances of the present invention. Such agents may include, but are not limited to, 5-chloro-2-(2,4-dichlorophenoxy)-phenol, commonly referred to as triclosan, chlorhexidine, alexidine, hexetidine, sanguinarine, benzalkonium chloride, salicylamide, domiphen bromide, cetylpyridium chloride (CPC), tetradecylpyridinium chloride (TPC); N-tetradecyl4-ethyl 1pyridinium chloride (TDEPC); octenidine; delmopinol, octapinol, and other piperidino derivatives, niacin preparations; zinc/stannous ion agents; antibiotics such as AUGMENTIN, amoxyicillin, tetracycline, doxycyline, minocycline, and metronidazole; and analogs and salts of the above antimicrobial antiplaque agents.

Anti-inflammatory agents can also be present in the oral care substances or compositions of the present invention. Such agents may include, but are not limited to, non-steroidal anti-inflammatory agents or NSAIDs, such as ketorolac, flurbiprofen, ibuprofen, naproxen, indomethacin, aspirin, ketoprofen, piroxicam and meclofenamic acid.

Nutrients may improve the condition of the oral cavity and can be included in the oral care substances or compositions of the present invention. Examples of nutrients include minerals, vitamins, oral nutritional supplements, enteral nutritional supplements, and mixtures thereof.

An individual enzyme or combination of several compatible enzymes can also be included in the oral care substance or composition of the present invention. Enzymes are biological catalysts of chemical reactions in living devices. Enzymes combine with the substrates on which they act forming an intermediate enzyme-substrate complex. This complex is then converted to a reaction product and a liberated enzyme which continues its specific enzymatic function.

Enzymes provide several benefits when used for cleansing of the oral cavity. Proteases break down salivary proteins which are absorbed onto the tooth surface and form the pellicle; the first layer of resulting plaque. Proteases along with lipases destroy bacteria by lysing proteins and lipids which form the structural component of bacterial cell walls and membranes. Dextranases break down the organic skeletal structure produced by bacteria that forms a matrix for bacterial adhesion. Proteases and amylases, not only present plaque formation, but also prevent the development of calculus by breaking-up the carbohydrate protein complex that binds calcium, preventing mineralisation,

Enzymes useful in the present invention include any of the commercially available proteases, glucanohydrolases, endoglycosidases, amylases, nutanases, lipases and mucinases or compatible mixtures thereof. Preferred are the proteases, dextranases, endoglycosidases and multanases, most preferred being papain, endoglycidase or a mixture of dextranase and mutanase.

Other materials that can be used with the present invention include commonly known mouth and throat products. These products include, but are not limited to anti-fungal, antibiotic and analgesic agents.

Antioxidants are generally recognised as useful in compositions such as those of the present invention. Antioxidants that may be included in the oral care composition or substance of the present invention include, but are not limited to Vitamin E, ascorbic acid, Uric acid, carotenoids, Vitamin A, flavonoids and polyphenols, herbal antioxidants, melatonin, aminoindoles, lipoic acids and mixtures thereof.

Histamine-2(H-2)receptor antagonist compounds (H-2 antagonists) may be used in the oral care composition of the present invention. As used herein, selective H-2 antagonists are compounds that block H-2 receptors, but do not have meaningful activity in blocking histamine-1(H-1) receptors.

The oral healthcare substance of the present invention can be in a dry form e.g. solid particles, or in a fluid form e.g. in the form of a viscous liquid, paste, gel, solution or any other suitable form. Preferably, the substance is in the form of a gel, which may be an aqueous or non-aqueous (e.g. based on glycerol) gel, and may also include a gelling or thickening agent. It may be necessary to add additional gelling agents in the formula to help the active ingredients adhere to the tissues of the oral cavity. Suitable agents include both polymers with limited water solubility as well as polymers lacking water solubility. Suitable gelling agents useful in the present invention include carboxy-polymethylene; carboxymethyl cellulose, carboxypropyl cellulose, polyaxamers, carrageenan, Veegum, carboxyvinyl polymers, and natural gums such as gum karaya, xanthan gum, guar gum, gum arabic, gum tragacanth, and mixtures thereof. A preferable gelling agent for use in the present invention is carboxypolymethylene, obtained from B. F. Goodrich Company under tradename Carbopol®. Particularly preferable Carbopols include Carbopol 934, 940, 941, 956 and mixtures thereof.

If the oral care substance is an aqueous gel, the water present in the gel compositions should preferably be deionised and free of organic impurities. A pH adjusting agent may also be added to optimise the storage stability of the gel and to make the substance safe for the oral tissues. These pH adjusting agents, or buffers, can be any material which is suitable to adjust the pH of the oral care substance. Suitable materials include sodium bicarbonate, sodium phosphate, sodium hydroxide, ammonium hydroxide, sodium stannate, triethanolamine, citric acid, hydrochloric acid, sodium citrate, and combinations thereof. The pH adjusting agents are added in sufficient amounts so as to adjust the pH of the substance or composition to a suitable value, e.g. about 4.5 to about 11 (The material Proxigel has pH ca. 4.7-5.2), preferably from about 5.5 to about 8.5, and more preferably from about 6 to about 7. pH adjusting agents are generally present in an amount of from about 0.01% to about 15% and preferably from about 0.05% to about 5%, by weight of the oral care substance.

An additional carrier material may also be added to the oral care substance or composition. These materials are generally humectants and include glycerin, sorbitol, polyethylene glycol and the like. The oral healthcare substance may comprise the substance itself, together with one or more substance enhancers, for example catalysts and/or potentiators to modify the release and/or activity of the substance. The device of the invention may additionally comprise additional substances such as flavours, colours etc, which may for example be deposited onto the surface of the strip or impregnated into the bulk of the strip in a manner analogous to the above described oral healthcare substance and adhesive, mutatis mutandis.

The oral healthcare substance is preferably a tooth whitening substance, preferably a peroxide-containing gel. Suitable gels may be based on glycerol containing a peroxide such as hydrogen peroxide or an organic peroxide. A suitable gel is that disclosed in U.S. Pat. No. 3,657,413, for example that sold under the trade mark PROXIGEL™ by The Block Drug Company (USA) (since acquired by GlaxoSmithKline plc). Other suitable peroxide-containing gels are for example disclosed in the art references cited above. The effectiveness of peroxide materials may be enhanced by means of a catalyst, i.e. a two-component peroxide-catalyst system. The strip may have the oral healthcare substance deposited upon its surface. For example a gel may be deposited directly as a layer on a surface of a plastically deformable strip, e.g. a wax strip, as described above. Alternatively a gel may be absorbed into the above-mentioned carrier layer, or impregnated into the bulk of the strip material, or deposited between layers of a multiple layered strip e.g. as described above.

Methods of depositing substances upon the surfaces of strip materials as described above are known, for example printing, e.g. silk screen printing, passing between impregnated rollers, dosing, a pump and nozzle, spraying, dipping etc. Methods of impregnating substances into the bulk of a strip material are also known, for example admixing the substance into the strip material and then forming the strip, or exposure of the strip to the substance under conditions which cause the substance to be impregnated into the strip. Alternatively example the strip material may be a foam material, particularly an open-cell foam material, and the substance may be impregnated into the strip material by introducing the substance into the cells of the foam. Some suitable technologies for deposition and impregnation of substances onto or into strip materials are known, for example from the transdermal patch art. The substance may subsequently be released from the strip by the action of moisture in the oral cavity dissolving the substance, or the effect of chemicals, enzymes etc, e.g. saliva amylases, present in the oral cavity.

The device of the invention may be marked with one or more visible symbol, e.g. text matter, a trade mark, a company logo, an area of colour, or an alignment feature such as a visible line or notch etc. to assist the user in applying the device to the teeth in a proper alignment. Such an alignment feature may for example comprise a symbol to show the user which way up the device should be whilst applying the device to the teeth, or which of a pair of the devices is intended for the upper teeth and which for the lower teeth. In this way the device may be made more visually attractive and/or easier to use. Such symbol(s) may be applied by conventional printing processes, e.g. silk screen printing, inkjet printing etc. to the surface of the plastically deformable material opposite to the surface on which is attached the layer of an absorbent material.

If such a visible symbol is applied to this surface a cover layer may be applied over the symbol, for example to protect it. This cover layer may be transparent or translucent to allow visible symbols to be seen through this layer.

Such a cover layer may be applied to the plastically deformable material by pressing, e.g. rolling, the material of the cover layer in contact with the plastically deformable material.

It may be preferable to provide an impermeable backing layer opposite the surface in contact with the tooth surface.

The invention may be realised in various forms.

A preferred form of the present invention comprises a strip of a plastically deformable material, to at least one surface of which is attached a layer of an absorbent material, with an oral healthcare substance on the layer of absorbent material.

In this preferred form the plastically deformable material may comprise the above-mentioned dental wax, and may suitably be 0.2-1.0 mm thick, preferably 0.2-0.5 mm thick, most preferably ca. 0.4-0.5 mm thick. Known types of dental wax can be rolled to such a thickness and formed into a strip shape.

The absorbent material in this preferred form is preferably a fabric, woven or non-woven, preferably non-woven. A suitable non-woven fabric is a polypropylene-viscose blend. A suitable thickness for the layer of absorbent fabric is 0.05-0.2 mm, preferably 0.075-0.125 mm, especially ca. 0.1 mm. Suitably the total thickness of such a wax plus adsorbent material strip may be ca. 0.7 mm. The layer of absorbent fabric may be attached to the strip of plastically deformable material by known methods, for example by bringing the fabric into contact with the surface of the strip and applying pressure, e.g. in a calendering process. In this way the plastically deformable material may be forced among the fibres of the absorbent fabric to thereby generate a bond.

In this preferred form the oral healthcare substance is preferably the above-mentioned peroxide-containing gel. Typically the gel may be applied to the absorbent fabric using conventional methods for example applying by means of a roller. Such a gel can soak into the adsorbent material and can be completely absorbed by the material, or some may remain unabsorbed as a surface layer. It is found that the absorbent fabric helps to retain the substance on the device, because a substance such as the above-mentioned tooth whitening gel can be absorbed into the absorbent fabric. The above-mentioned preferred thickness of fabric is found suitable to retain a sufficient quantity of such a gel on the device. The absorbent fabric also helps to prevent the substance, if it is a fluid gel, from being squeezed out from between the strip and the teeth surfaces when the device is pressed against the teeth. Typically a loading of 250-750 mg of such a peroxide-containing gel substance may be used, preferably 250-500 mg. It should be noted that not all of the oral healthcare substance loaded onto the device might actually ultimately contact the oral surfaces, and in a test using a peroxide-containing gel ca. 70% of substance loaded was actually transferred to tooth surfaces.

In this preferred form the strip of plastically deformable material, e.g. the wax, may be marked with one or more visible symbol as mentioned above, typically applied to the surface of the plastically deformable material opposite to the surface on which is attached the layer of an absorbent material. When such a visible symbol is applied it is preferred to apply a cover layer as mentioned above over the symbol. This cover layer preferably comprises the same material, e.g. the wax, as the layer of plastically deformable material so that the symbol is in effect embedded in the plastically deformable material, or sandwiched between the strip and the cover layer of wax material.

Such a cover layer may be applied to the plastically deformable material by pressing, e.g. rolling, the material of the cover layer in contact with the plastically deformable material.

A typical process for making the above-mentioned preferred form of the device of the invention using a dental wax as provided in the form of a sheet or reel as the plastically deformable material, may involve the following steps.

If necessary, cleaning the surface of the wax material. As provided dental wax may have a surface contamination of for example coconut oil, and typically wiping the surface with for example a non-woven cloth may be suitable to clean the surface. A continuous extrusion process may be used to prepare wax substantially free of surface contamination.

A visible symbol may be printed upon a first surface of a first sheet of the wax material. Suitably one or more ink jet printer may be used for this, using a suitable ink, preferably an ink which is orally acceptable.

A second, cover sheet of the wax material may be laminated to the first surface of the first sheet, to sandwich the printed symbol between the first and second sheets, and in effect to embed the printed symbol in the laminated sheets of dental wax. This may be done by pressing, e.g. rolling the first and second sheets together between rollers at a suitable pressure, and elevated temperature if found necessary (e.g. rollers at a surface temperature ca. 85° C., with a speed of 200 mm/sec), which can easily be determined by those skilled in the art.

The laminated sheets may then be sized by compressing them, e.g. between rollers, to compress them to a suitable thickness, e.g. as mentioned above.

The layer of absorbent fabric may then be attached to the laminated wax strips by known methods, for example by bringing the fabric into contact with a surface of the first sheet opposite to the first surface, and applying pressure. For example the laminated sheets of wax and the fabric may be rolled together between rollers. In this way the plastically deformable material may be forced among the fibres of the absorbent fabric to thereby generate a bond.

In the above-mentioned steps where the wax sheets are compressed between rollers it may be necessary to provide a sheet of a “sacrificial” material e.g. a paper between the wax sheet and a roller to prevent the wax sticking to the roller. For example a preferred way of providing the wax may be to continually cast it as a sheet on a release paper carrier.

The sheet of laminated material may then be cut to shape, for example using a die cutter. The oral healthcare substance may then be applied to the layer of absorbent material, using a conventional method such as applying by means of a roller, or preferably using a delivery pump and nozzle.

In another form of the invention, the substance and an adhesive material may be deposited in separate discrete locations in relation to the strip surface.

The adhesive may be any adhesive which may be used to stick materials to the tooth surface or to a surface of the oral cavity such as a gum or other skin surface or mucous membrane, so that part of the strip on which the substance is deposited or into which it is impregnated may contact the tooth surface, and thereafter be easily removable by the user. Suitable adhesives include skin-, gum- and muco-adhesives, and should be able to withstand the moisture, chemicals and enzymes of the oral environment for long enough for the oral healthcare substance to take effect, but may be soluble and/or biodegradable thereafter. Suitable adhesives may for example comprise hydrophobic and/or non-water soluble polymer devices. Suitable adhesives may for example include pressure and moisture sensitive adhesives, e.g. dry adhesives which become tacky upon contact with the mouth environment, e.g. under the influence of moisture, chemicals or enzymes etc. in the mouth. Suitable adhesives are known, and for example include natural gums, synthetic resins, natural or synthetic rubbers, and various other tacky substances of the kind used in known adhesive tapes. A suitable adhesive material has long been known from U.S. Pat. No. 2,835,628. As mentioned above the strip material may have wells or dimples in its surface, or may have a sponge-like surface, to contain an adhesive material.

Various embodiments of this form of the invention are discussed below.

In one embodiment the oral healthcare substance and adhesive may be deposited on the surface of the strip in respective spatially separated places on the surface. For example the adhesive may be deposited in places on the strip surface that enable part of the strip to stick to an oral surface adjacent to a tooth surface, e.g. a gum surface, so that another part of the strip on which the substance is deposited or into which it is impregnated may contact the tooth surface.

Alternatively the adhesive and substance may be spatially separated but both in locations that enable the adhesive and substance to contact the same type of tissue, e.g. tooth or gum surface. For example the oral healthcare substance and adhesive may be deposited on the surface in respective discrete spots or patches on the surface. Such patches or spots should be relatively small so that for example the oral healthcare substance does not exercise its effect patchily on the tooth surface. For example the oral healthcare substance and adhesive may be deposited on the surface in respective discrete lines on the surface, for example respective parallel lines of the adhesive and oral healthcare substance. For example the oral healthcare substance may be deposited in one or more patch bordered partly or completely surrounded by a border of the adhesive. For example such a patch of oral healthcare substance may comprise a single large patch covering substantially the entire surface of the strip, and bordered along its long edges by a line of the adhesive, or completely surrounded by a border of adhesive. Vice versa for example the adhesive may be deposited in one or more patch bordered partly or completely surrounded by a border of the oral healthcare substance. In such embodiments the oral healthcare substance may be deposited in a release composition containing the substance in a solid, polymer or gel matrix from which it may be leached out under the action of water, chemicals and/or enzymes in the oral environment. Suitable technologies for such release compositions are known, for example using polyvinyl alcohol or derivatives thereof, celluloses such as hydroxypropylmethyl celluloses etc. The adhesive may for example be a hydrophobic or non-water soluble adhesive, and if such an adhesive is used as a border around a patch of oral healthcare the adhesive border can form a barrier hindering escape of the substance via the edges of the strip.

In another embodiment the substance and/or adhesive may be encapsulated. Encapsulation may for example be in micro-capsules, or macro-capsules. Methods of micro-encapsulation are known, for example in which a droplet of a substance is enclosed in a liquid phase within a layer of an encapsulation material, and then separated from the liquid. Such capsules may be deposited on or adjacent the surface of the sheet, and may for example be burst physically or chemically, e.g. by pressure e.g. as the strip is applied to the tooth surface or by subsequent bite action, by breaching of the capsule wall under the action of the temperature, moisture, pH, chemicals or enzymes in the mouth environment etc. For example respective capsules of oral healthcare substance and adhesive may be attached to the surface of the strip, e.g. by means of a second adhesive or by embedding the capsules in the strip material. For example a thin layer of the adhesive may be deposited on the surface of the strip, and capsules of the oral healthcare substance may be embedded at least partly if not completely within this adhesive layer, or may sit upon the surface of this adhesive layer. For example, vice-versa to the last described construction, a thin layer of a release composition of the oral healthcare substance may be deposited on the surface of the strip, and capsules of the adhesive may be embedded at least partly if not completely within this layer of composition. For example such capsules may be in the form of microbubbles of a bubble film material bonded to the surface of the strip, so that for example a wall of the bubble may comprise the strip itself.

In another embodiment the substance and/or adhesive may be provided in granules, e.g. pellets or micropellets, which may release their content under the influence of the mouth environment, for example moisture, chemicals or enzymes in the mouth, and may be coated to achieve this release. Methods of granulation and pelletizing are known, as are coating polymers such as the known Eudragit™ polymers which dissolve at specified pH. Such granules may be deposited on or adjacent the surface of the strip. For example respective granules of oral healthcare substance and adhesive may be attached to the surface of the strip, e.g. by means of a second adhesive or by embedding the granules in the strip material. For example a thin layer of the adhesive may be deposited on the surface of the strip, and granules of the oral healthcare substance may be embedded at least partly if not completely within this adhesive layer. For example, vice-versa to the last described construction, a thin layer of a release composition of the oral healthcare substance may be deposited on the surface of the strip, and granules of the adhesive may be embedded partly or completely within this layer of composition.

For example capsules and/or granules of adhesive and substance may be located substantially uniformly over the strip surface, or alternatively respective capsules and/or granules of adhesive or substance may be situated at separate respective locations on the surface of the strip. Alternatively one of an adhesive or substance may be provided in capsules or granules, and the other may be deposited on or impregnated into the strip. For example adhesive may be positioned at parts of the strip that contact an oral surface adjacent to a tooth surface e.g. deposited, impregnated or in capsules or granules, and the part(s) of the strip that contact the tooth surface may have the substance thereon, e.g. deposited, impregnated or in capsules or granules.

In embodiments in which the oral healthcare substance is provided in the form of capsules or granules deposited on a surface of the strip, e.g. as described above, the capsules or granules may be covered by a porous membrane layer, e.g. of a non-woven fabric material as described above. Such a membrane layer may help to retain the capsules or granules on the surface of the strip, and in the case of capsules may also help to retain capsule casing debris when the capsules have opened to release their content, whilst allowing active material content to pass through. Such an embodiment may for example be made by depositing the granules and/or capsules on the surface of the strip, and then laminating the membrane layer onto the strip over the capsules. For example capsules and/or granules may be deposited in a region on the strip, and the membrane layer may be bonded to the strip around the region.

Potentiators and/or catalysts as mentioned above may for example be provided in other capsules or granules which release their contents in the mouth. Alternatively such potentiators and/or catalysts may be provided externally to such capsules or granules of substance, e.g. as an outer coating on the capsules or granules, or deposited on or impregnated in the strip material adjacent to the capsules or granules, e.g. in a layer of material to which the capsules or granules are attached or in which they are embedded.

In another embodiment the adhesive and oral healthcare substance may be deposited in separate discrete layers on the surface of the strip. For example a layer of the oral healthcare substance, e.g. in the form of a release composition may be deposited relatively proximal to, e.g. adjacent to and in contact with the surface, and a layer of the adhesive may be deposited relatively distal from the surface e.g. adjacent to and in contact with an underlying layer of oral healthcare substance. In such a construction the adhesive may stick the strip to the tooth surface, and the substance may pass through the adhesive layer, for example as the adhesive layer becomes permeable under the influence of the mouth environment. The adhesive layer may in such a construction have one or more hole passing through the layer to facilitate the passage of the substance through the adhesive layer. Alternatively for example a layer of the adhesive may be deposited relatively proximal to, e.g. adjacent to and in contact with the surface, and a layer of the of the oral healthcare substance, e.g. in the form of a release composition may be deposited relatively distal from the surface e.g. adjacent to and in contact with an underlying layer of adhesive. In such a construction the layer of oral healthcare substance may need one or more hole passing through the layer to facilitate the passage of the adhesive through the layer of oral healthcare substance. In the above constructions the passage of material from the underlying layer may be facilitated by pressure as the strip is applied to the tooth surface.

In another embodiment the strip material may be inherently adherent to a tooth surface. For example the strip material may adhere to the tooth surface by surface tension under the influence of moisture in the mouth. For example the strip material may be a hydrophilic polymer having for example muco-adherent properties. For example the film may have impregnated or dispersed within it an adhesive material, suitably a dry adhesive material that becomes tacky in contact with moisture, chemicals or enzymes within the mouth environment. Alternatively example the strip material may be a foam material, particularly an open-cell foam material, and the adhesive may be impregnated into the strip material by introducing the substance into the cells of the foam. The oral healthcare substance may be deposited upon a surface of such a strip, for example in spots, patches, lines, or as a layer e.g. a porous or perforated layer on the surface through which the adhesive may pass.

According to further forms of the invention, mechanical adhesive means may also be used to provide an adhesive function, used either alone or in combination with any other adhesive device disclosed herein. In preferred embodiments mechanical adhesion between the strip and tooth or other oral surface is provided by the strip comprising a plastically deformable material, particularly the above-mentioned wax, which can be plastically deformed by the user to conform the strip to the contours of the tooth or other oral surface, particularly fitting into convavities and spaces between teeth, and so adhere thereto by mechanical gripping. A layer of an oral healthcare substance such as the above-described gel materials may be deposited thereon. Such gripping may be enhanced by e.g. surface effects between the strip and the surface such as formation of a partial vacuum or surface tension effects For example the strip may have anchors on its surface, positioned at approximately the spacings of gaps between teeth, and these anchors may fit into the gaps between the teeth. For example the surface of the strip which is to contact the tooth surface may be provided with micro-suckers, that is a plurality of small cavities in the surface of the strip which can be pressed onto the tooth surface to drive air out therefrom, and thereby create a partial vacuum, so that the strip is thereafter held on the tooth surface by air pressure. Such anchors or micro-suckers may be located on the surface of a strip which is to contact the tooth surface. Such a strip may for example be stretchable, for example softening under the action of moisture, chemicals or enzymes in the mouth so that it can be adjusted to the spacings of gaps between an individual user's teeth. Another form of “mechanical” adhesion may be provided by a strip material which shrinks in contact with the tooth surface, for example in contact with moisture, chemicals or enzymes in the mouth, or any other feature of the mouth environment, so that the shrunken strip can physically grip the surface of the tooth.

Suitable shapes of the device of the invention, for example the above-mentioned preferred form of the device, will now be discussed. The device of the invention is suitably of elongate shape, of a length sufficient that when placed against the front surface of the teeth of a user it extends across a plurality of teeth. Suitably the device is sufficiently long to extend at least the user's canine teeth, for example to the pre-molar teeth.

The device is suitably of sufficient width that when placed against the user's teeth it extends from the gumline at least to the crowns of the front teeth distant from the gumline. Suitably the width is such that in an unfolded state the strip has an unfolded width greater than the height of the teeth from the gumline to the crown, and at least part of the strip may be folded about a substantially longitudinal fold axis so as to bend or fold over the crowns and contact the crowns and rear surfaces of the user's teeth, i.e. so that in cross section the folded part of the device is substantially of a “U” or “V” shape, with two limbs linked at a fold axis each limb with an inward surface facing into the “bite” of the “U” or “V”, with the oral healthcare substance present on this inward, tooth-contacting surface. Such a strip can be applied to a tooth so that the inward facing surface of a first limb can be applied to one surface of a tooth, e.g. a surface that faces into the interior of the mouth, and the inward facing surface of a second limb can be applied to an opposite surface of the tooth, e.g. a surface that faces outwardly, e.g. a front surface of the tooth.

If necessary, an adhesive material may be deposited on the first inward facing surface and the oral healthcare substance may be deposited on the second inward facing surface. However in the above mentioned preferred form of the device it is believed that the physical, i.e. mechanical conformation of the plastically deformable wax to the shape of the teeth, e.g. fitting into the interdental spaces etc., causes sufficient attachment of the preferred form of the device to the user's teeth. Pressure may be applied by the user to achieve this. When the preferred peroxide-containing gel is used as the oral healthcare substance with the preferred form of the device then this gel may in fact act as a slimy lubricant reducing adhesion between the device and a tooth surface, rather than as an adhesive.

The device of the invention may be of various shapes, depending upon whether it is to be applied to the upper or lower teeth. In general, for both upper and lower teeth the device may be substantially rectangular or trapezoidal, for example with rounded corners and/or ends.

Suitably to be applied to the upper teeth the device may be substantially rectangular with concavely curved long sides. To be applied to the lower teeth the device may be substantially rectangular with convexly bowed long sides, or of a generally rectangular shape but with a concave curved long side or a concave indentation in a long side.

Suitably, in particular for application to the upper teeth the device may have a tab extending from a long side. For example such a tab may be substantially rectangular. Such a tab may be used to assist the user in manipulating the device and applying it to the tooth surfaces.

The dimensions of the device will in practice be determined principally by the typical dimensions of a user's teeth. Typically the device may be ca. 4-10 cm long×ca.1.0-3 cm wide, typically ca. 7-8 cm long×ca.2-2.5 cm wide. This is found to be a suitable width to enable the strip to be applied to both the front and rear surfaces of the user's teeth, by folding the strip into a “U” shape over the crowns of the teeth. These sizes are suitable for a loading of a peroxide-containing gel as mentioned above of ca. 200-750 mg.

If the substance is impregnated into the strip material so as to be released from the strip material onto the tooth surface, variants of the above-described options may be used.

Typical use of the device of this invention will now be described. The device of this invention may be applied manually to a user's tooth surface(s), for example using the user's fingers or using an applicator device to position the strip on or adjacent to the tooth surface. The device is applied by the user to his/her teeth, with the length dimension of the strip aligned with the line of the user's teeth, the substance-bearing surface in contact with the front surface of the teeth, and the device is pressed against the front surface of the teeth.

When using the preferred form of the device, the plastic deformation of the strip material causes the device to become shaped to the profile of the teeth, and to fit into the gaps between the teeth. If the device has a width greater than the length of the teeth it may be folded over the distant ends of the teeth to contact the rear surfaces of the teeth and be pressed against the rear surface, thereby becoming shaped to the rear surfaces of the teeth. A tab, if present, can assist the user in manipulating the device into place.

The plastic deformation of the preferred form of the device causes the device to remain in its deformed shape after the deforming pressure has ceased, until the device is removed again, e.g. by the user. The adapting of the device to fit the shape of the teeth, combined with any folding of the device over the distant ends to contact the rear surfaces of the teeth, is the principal force holding the device in place against the teeth. The device may also be held against the teeth by one or more other forces in addition to the grip provided by this plastic deformation, for example surface tension from mouth fluids such as saliva, the pressure of adjacent mouth surfaces such as the lips against the device.

The device is left in contact with the user's teeth for a sufficient length of time for the oral healthcare substance to have its effect. The period of use will depend upon the particular user, convenience, the state of the user's teeth, the healthcare effect desired, e.g. degree of whitening required etc. This period may for example be as short as 10 minutes, or may be longer for example 2 hours, and the device may be applied for plural sessions each day, e.g. two 30 minute sessions per day. The use of the device may be repeated for a similar length of time over a course of several days, e.g. 7 to 14 days until a desired extent of the oral healthcare effect, e.g. tooth whitening, has been achieved. Whilst the device is in contact with the teeth, the oral healthcare substance is held in immediate contact with the teeth surfaces and is protected from removal as a result of washing with mouth fluids or contact by adjacent mouth surfaces by the covering layer of the plastically deformable material. After use the device may simply be removed from the teeth and disposed of, and the user may rinse his/her mouth, and/or brush his/her teeth, to remove residual healthcare substance if desired. By separation of the adhesive and active functions, especially by use of the gel materials described which have little or no adhesive effect but use the mechanical adhesion achieved by mechanical gripping of the teeth by for example a plastically deformable wax material, it is particularly easy to rinse the substance off the teeth and oral tissues after use.

The device may be supplied for use with a protective cover film over the oral healthcare substance to protect it from contamination and/or loss. This film may be an easy to peel off film.

Individual devices of the invention may be supplied contained for example in sachets, e.g. correspondingly sized generally flat envelopes, and suitably such sachets can be opened at one face thereof so that the device contained therein can be removed without any longitudinal sliding motion which might scrape off the substance. Suitably a sufficient number of such device-containing sachets may be contained in an outer pack to enable a user to complete a course of use. Such a pack, or individual sachets, may be marked with instructions for use, or the pack may contain an instruction leaflet. The invention will now be described by way of example only with reference to the accompanying figures which show:

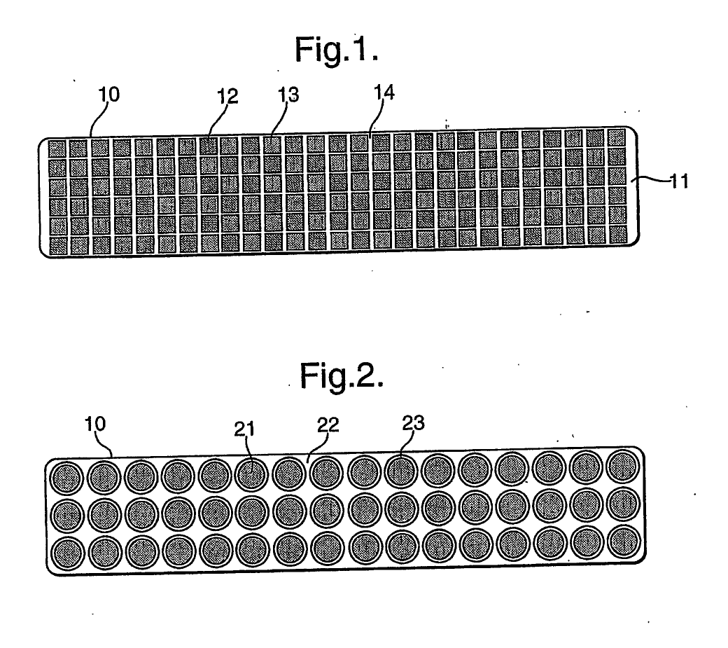

FIG. 1 A plan view of the surface of a strip having patches of oral healthcare substance and patches of adhesive deposited thereon.

FIG. 2 A plan view of the surface of a strip having patches of oral healthcare substance surrounded by an adhesive deposited thereon.

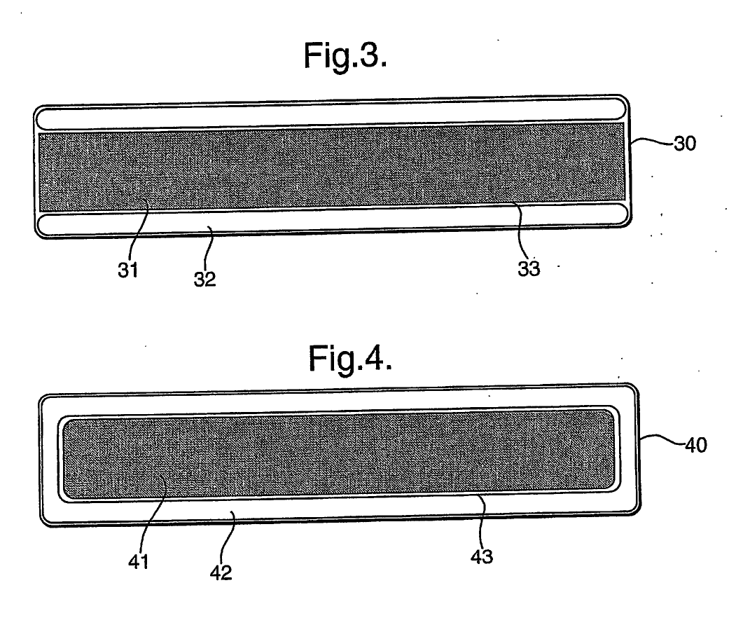

FIG. 3 A plan view of the surface of a strip having a patch of oral healthcare substance and lines of adhesive deposited thereon.

FIG. 4 A plan view of the surface of a strip having a patch of oral healthcare substance and a surrounding border of adhesive deposited thereon.

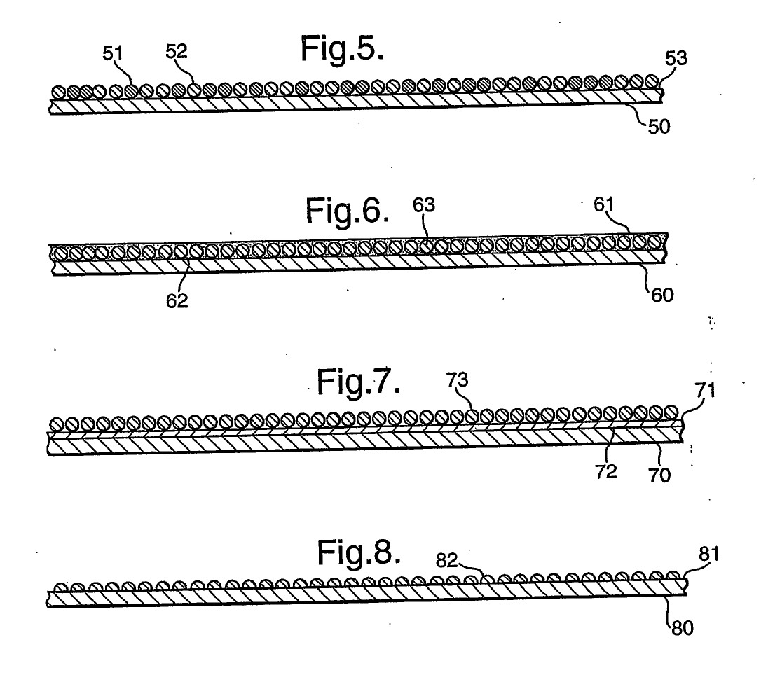

FIG. 5 A longitudinal section of a strip having encapsulated oral healthcare substance and adhesive deposited thereon.

FIG. 6 A longitudinal section of a strip having encapsulated oral healthcare substance embedded in a layer of adhesive deposited thereon.

FIG. 7 A longitudinal section of a strip having encapsulated oral healthcare substance and a layer of adhesive deposited thereon.

FIG. 8 A longitudinal section of a strip having microbubbles suitable for encapsulation of oral healthcare substance and/or adhesive.

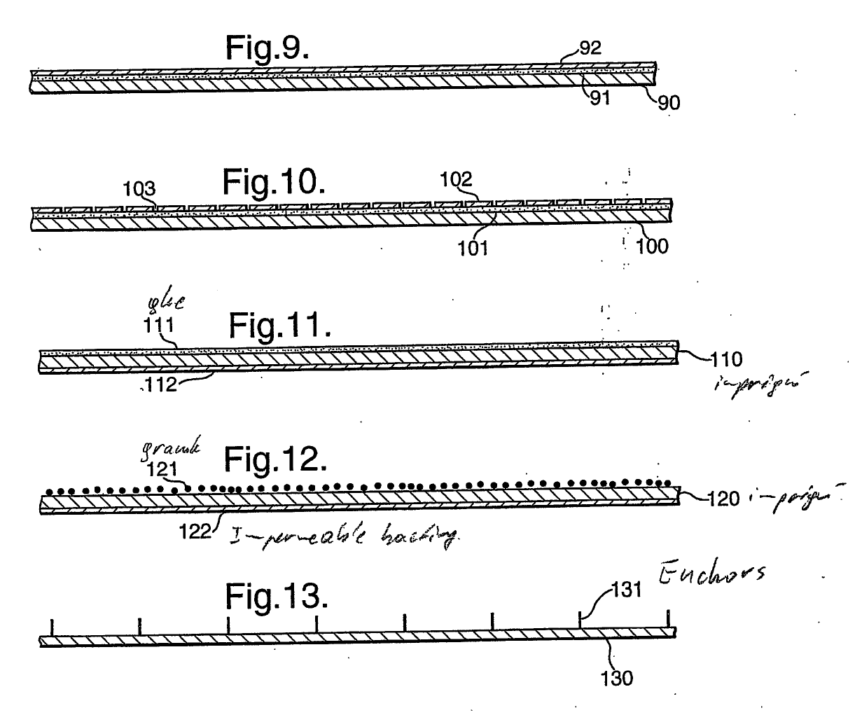

FIGS. 9-10 show longitudinal sections of strips having layers of adhesive and oral healthcare substance deposited thereon.

FIGS. 11-12 show longitudinal sections of strips having an oral healthcare substance impregnated therein.

FIG. 13 shows longitudinal sections of a strip having small anchors thereon.

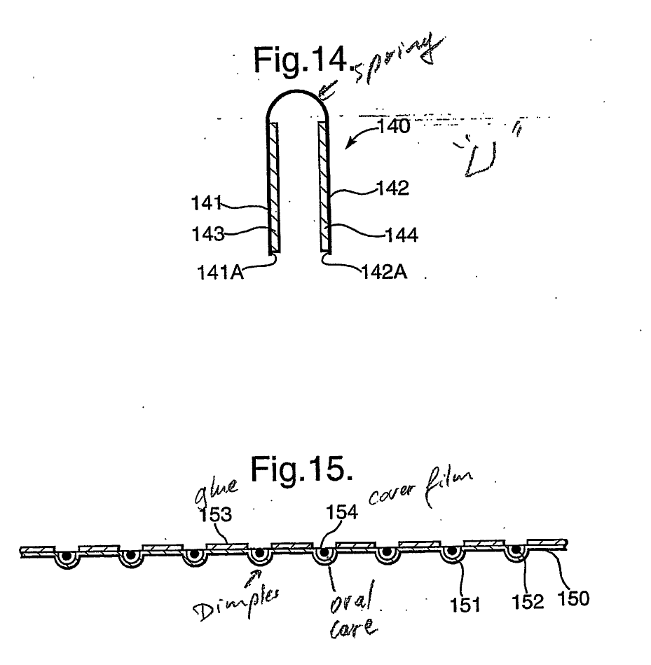

FIG. 14 shows a cross section of a strip with a “U” shaped cross section.

FIG. 15 shows a longitudinal section of a strip having small dimples suitable for containing an oral healthcare substance therein.

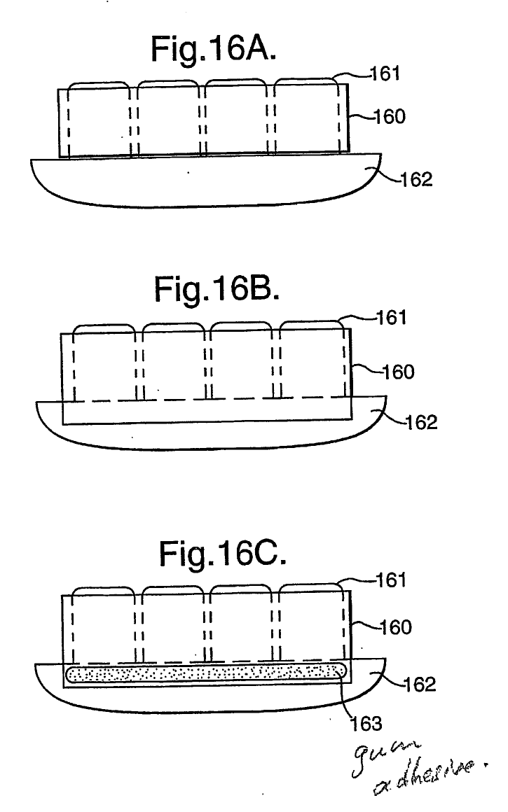

FIG. 16 shows a device as shown in FIGS. 1-15 in use.

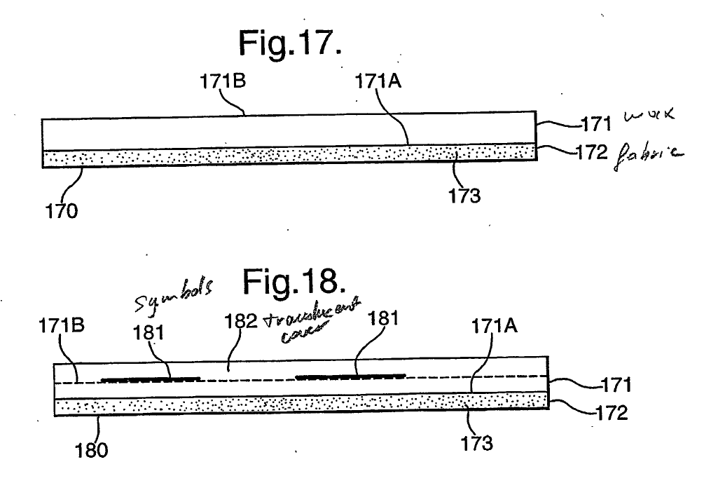

FIG. 17 shows a cross section through a preferred form of the device of this invention

FIG. 18 shows a cross section through another preferred form of this invention.

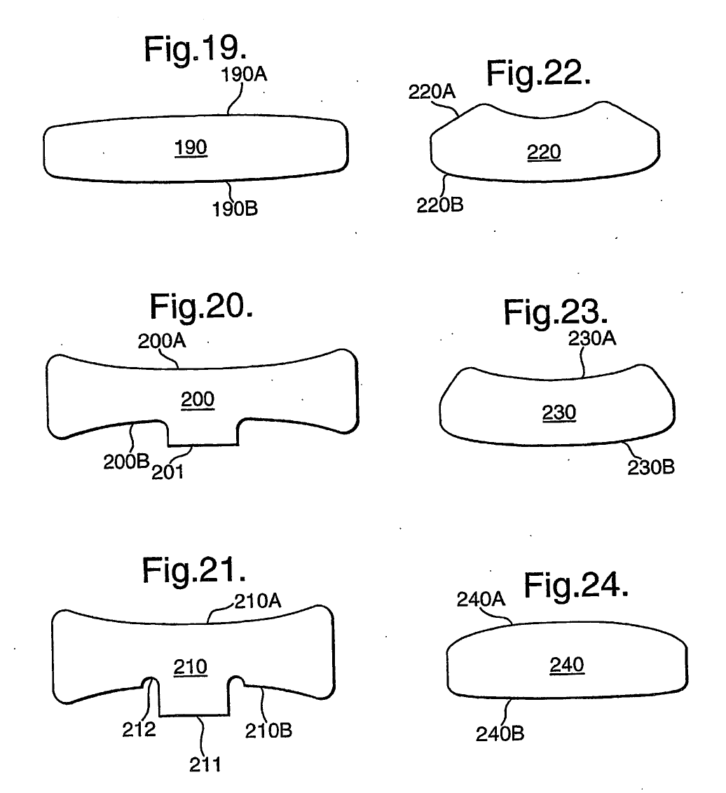

FIGS. 19-21 show plan views of a preferred form of the device of this invention suitable for the upper teeth

FIGS. 22-24 show plan views of a preferred form of the device of this invention suitable for the lower teeth

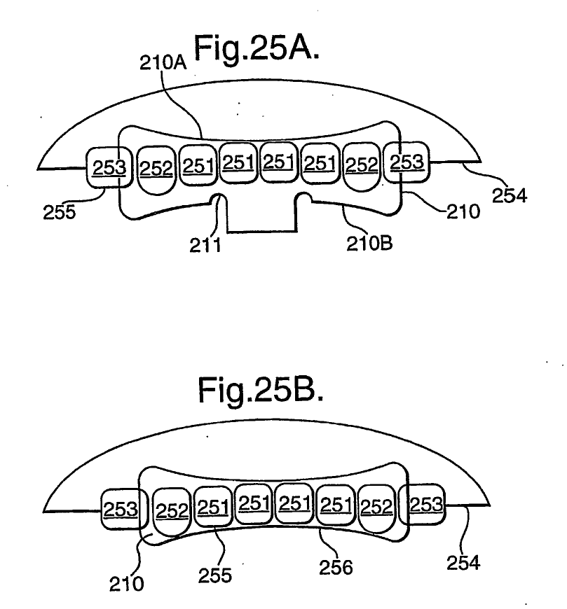

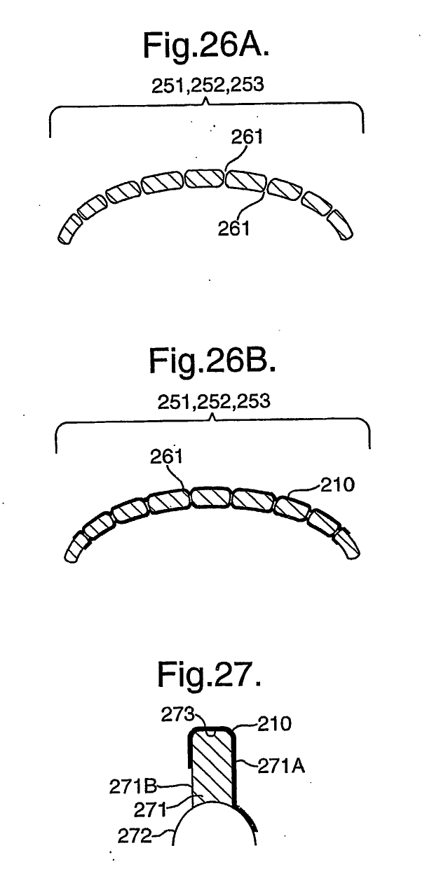

FIGS. 25 and 26 show the device of FIGS. 17-23 in place on the user's teeth

FIG. 27 shows a section through a tooth with the device of FIGS. 17-23 in place.

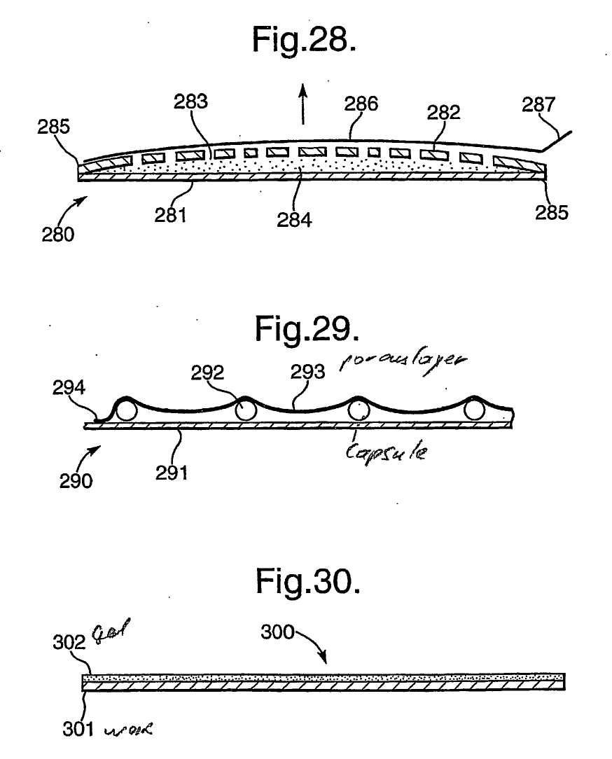

FIGS. 28-30 show further devices of the invention.

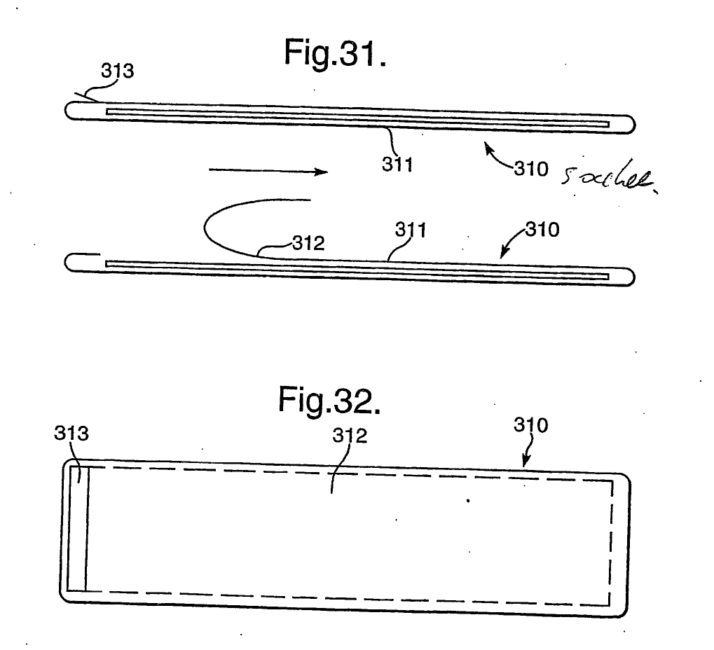

FIGS. 31 and 32 show a suitable package for the device of the invention.

Referring to FIG. 1 a strip 10 of a polymeric material such as thin flexible polyethylene, ca. 0.005-0.02 mm thick is shown in plan view. The strip 10 is generally rectangular with rounded corners, and has a width “W” approximately the length of human teeth, and a length “L” approximating to the width of several human teeth. The strip 10 can therefore be applied by gentle pressure to for example the front facing surface of the teeth and further pressure may then deform the strip 10 so that it accommodates itself to the contours of the user's teeth, with a surface 11 in contact with the teeth.

Patches of oral healthcare substance 12 and patches of adhesive 13 are deposited on the surface 11 of the strip 10 in respective spatially separated places on the surface 11, with a small gap 14 between them. The patches 12, 13 are relatively small (i.e. they are not drawn to scale in FIG. 1) so that the oral healthcare substance 12 does not exercise its effect patchily on the tooth surface. Although shown as rectangular the patches 12, 13 may be any shape, preferably a tessellating shape.

Referring to FIG. 2 the oral healthcare substance is deposited in a pattern of patches 21 of the surface of a strip 10, which are completely surrounded by a border of the adhesive 22, but again leaving a narrow gap 23 between the adhesive 22 and the patches 21.

Referring to FIGS. 3 and 4 a single large patch 31, 41 of oral healthcare substance is deposited on a surface of a strip 30, 40, covering substantially the entire surface of the strip 30, 40 (not drawn to scale). The patch 31 is bordered along its long edges by a line of the adhesive 32, and in FIG. 4 is completely surrounded by a linear border 42 of adhesive. There is a small gap 33, 43 between the patch of substance 31, 41 and the adhesive 32, 42. In FIGS. 3 and 4 the oral healthcare substance patches 31, 41 comprise a release composition containing the substance and from which it may be leached out in the mouth. The border 42 can form a barrier hindering escape of the substance via the edges of the strip.

Referring to FIG. 5, which shows a longitudinal section through a strip 50, the substance and adhesive are encapsulated in respective micro-capsules 51 of substance and 52 of adhesive, or macro-capsules attached by an adhesive (not shown) on a surface 53 of the strip 50. Capsules 51, 52 may be burst by pressure e.g. as the strip 50 is applied to the tooth surface or by subsequent bite action

Referring to FIG. 6, a strip 60 shown in longitudinal section has a layer of an adhesive 61 deposited on the surface 62 of the strip, and capsules 63 of the oral healthcare substance are embedded within this adhesive layer 63. Alternatively the layer 63 may comprise a layer of a composition of the oral healthcare substance deposited on the surface 62 of the strip, and from which the substance may be released, and the capsules 63 may contain the adhesive. In each case, as above the capsules may be burst by pressure.

Referring to FIG. 7, a strip 70 shown in longitudinal section has a layer of an adhesive 71 deposited on the surface 72 of the strip 70, and capsules 73 of the oral healthcare substance sit upon the upper surface of this layer and are thereby attached to the strip. When applied to the surface of a tooth, the adhesive 71 sticks the strip 70 to the tooth surface, and pressure bursts the capsules 73 to release the substance.

Referring to FIG. 8, a strip 80 is shown in longitudinal section, being a polyethylene film as above, and microbubbles 81 of a bubble film material are bonded to the surface of the strip 80, so that the lower wall of each bubble comprises the surface 82 of the strip 80 itself. Such microbubbles 81 may be used as the capsules in a construction analogous to FIG. 5, or may be embedded as shown in FIG. 6.

In all of the embodiments illustrated in FIGS. 5 to 8, pellets or micropellets may be used in place of the described capsules.

Referring to FIGS. 9 and 10, a strip 90,100 has an adhesive and an oral healthcare substance deposited on its surface in separate discrete layers, i.e. adhesive layer 91, 101, substance layer 92, 102. The layer 91, 101 of the oral healthcare substance is in the form of a release composition deposited under the layer 92, 102 of the adhesive. The adhesive layer 91, 101 sticks the strip 10 to the tooth surface, and the substance released from underlying layer 91,101 passes through the adhesive layer 92,102, for example as the adhesive layer 92,102 becomes permeable under the influence of the mouth environment. As shown in FIG. 10 the adhesive layer 102 has holes 103 passing through the layer 102 to facilitate the passage of the oral healthcare substance from layer 101 through the adhesive layer 102. Alternatively the underlying layer 91,101 may comprise the adhesive layer and the upper layer 92,102 may comprise a layer of the oral healthcare substance as a release composition, optionally also having the holes 103 passing through the layer 102 to facilitate the passage of the adhesive through the layer 102 of oral healthcare substance.

Referring to FIGS. 11 and 12, a strip 110, 120 is shown in longitudinal section. An oral healthcare substance is impregnated into the bulk of the strip 110,120 using a known method which allows the substance to leach out and be released in the mouth, e.g. using methodologies used with known buccal delivery devices. An adhesive material is deposited as a layer 111 over the surface of the strip 110,120, and this layer 111 may have holes therein through which the oral healthcare substance may pass. In FIG. 12, an encapsulated or granulated adhesive 121 is attached to the surface of the strip, e.g. using a second adhesive (not shown). Using the strips of FIGS. 11 and 12, these may be applied to a tooth surface and the adhesive 111 may stick the strip to the tooth surface, or in the case shown in FIG. 12, adhesive may be released from the capsules or granules 121 to thereby stick the strip to the tooth surface. The substance is then released from the strip 110,120. The strips 110,120 of FIGS. 11 and 12 are provided with an impermeable backing layer 112, 122.

Referring to FIG. 13, a strip 130 is shown in longitudinal section, which may have an oral healthcare substance deposited on or impregnated into it, and optionally an adhesive deposited onto it in any of the ways described above with reference to FIGS. 1 to 12. The strip 130 has small anchors 131 on its surface, positioned at approximately the spacings of gaps between teeth. These anchors 131 comprise small strips extending perpendicular from the surface 132 of the strip 130, and extending transversely across the width of the strip 130. These anchors 131 may fit into the gaps between the teeth. The strip 130 shown in FIG. 13 is stretchable, for example softening under the action of moisture, chemicals or enzymes in the mouth so that it can be adjusted to the spacings of gaps between an individual user's teeth.

FIG. 14 shows a longitudinal section through a strip 140 which is substantially “U” shaped in cross section, with two limbs 141, 142 linked at a fold. Each limb 141,142 has a surface 141A, 142A facing into the “bite” of the “U” so that the strip can be applied to a tooth (not shown) so that the inward facing surface 141A of a first limb e.g. 141 can be applied to the front surface of the tooth, and the inward facing surface 142A of the second limb 142 can be applied to the back surface of the tooth. An adhesive material 143 is deposited on the first inward facing surface 141A and the oral healthcare substance 144 may be deposited on the second inward facing surface 142A. Alternatively the strip 140 may have an oral healthcare substance impregnated into the bulk of the material of the strip 10. In either case the oral healthcare substance is released from the strip 140 to contact the front surface of the tooth. The material of strip 140 may be resilient so that without permanent deformation the strip 140 can be retained on the tooth by the inherent spring grip of the material.

FIG. 15 shows a strip 150 e.g. of a polyethylene material, having dimples 151 therein, and which contain an oral healthcare substance 152. Between the dimples 151 is deposited an adhesive material 153. The substance 152 may be fixed into dimples 151, and the dimples may be closed with a soluble cover film 154 which is breached in the mouth environment to release substance 152. In a vice versa construction, dimples 151 may contain the adhesive, and a release composition containing the substance may be deposited between the dimples 151.

FIG. 16 shows a view of a row of teeth 161, and the gum 162.

In FIG. 16A a strip 160 is shown attached by an adhesive (not shown) to the teeth surface, so that the effect of oral healthcare substance deposited on or impregnated into the strip 160 is exercised only or principally on the teeth.

In FIG. 16B a strip 160 is shown attached by an adhesive (not shown) to both the teeth 161 and gum 162 surfaces, so that the effect of oral healthcare substance deposited on or impregnated into the strip 160 is exercised on the surfaces of both the teeth 161 and gums 162.

In FIG. 16C a line 163 of a gum-adhesive is used by which the strip 160 sticks to the gum 162, with part of the strip 160 in contact with the teeth. Oral healthcare substance (not shown) is released from strip 160 and exercises its effect on the teeth 161 surfaces.

The view in FIG. 16 is looking from the underside direction of the strips as shown in FIGS. 1-14. Where occluded by strip 160 the teeth 161 are shown in dashed line.

FIGS. 17 to 25 relate to a preferred form of the device of this invention.

Referring to FIG. 17 a device 170 of the invention is shown in section. The device comprises a strip 171 of a plastically deformable material, being a dental wax (SW12 or SW13 sheet wax, obtainable from The Kindt-Collins Co., Cleveland, Ohio, USA), or waxes S & P wax 1, wax X1 or dental wax II from Strahl & Pitsch (favoured for its low odour) or a wax from Witco known as 13324. These waxes are available in sheets and can be used in this form or cast into a sheet with a thickness ca. 0.3 mm. Bonded to one surface 171A of the strip 171 is a layer 172 of an hydrophobic absorbent fabric, being a non-woven polypropylene/viscose blend, (grade HYN-35 non woven obtainable from BFF Non Wovens, Somerset, GB) . The layer 172 is ca. 0.1 mm thick. Opposite to surface 171A is a surface 171B, termed the “upper surface” of the strip 171. The layer 172 is ca. 0.5 mm thick in its uncompressed form and is bonded to strip 171 by starting with a strip 171 ca. 0.5 mm thick, bringing it into contact with the fabric, and squeezing the strip 171 and the fabric between heated rollers (e.g. rollers at a surface temperature ca. 85° C., with a speed of 200 mm/sec). The material of the strip 171 is thereby forced by pressure among the fibres of the fabric to bond them together. Deposited on the layer 172, and absorbed thereby, is a tooth-whitening gel 173 being the tooth whitening gel substance PROXIGEL™ sold by The Block Drug Company. The gels disclosed as Examples 1 or 2 of U.S. Pat. No. 3,657,413 are also suitable. The gel 173 is applied to the fabric 172 by for example using a roller or a brush, and is mostly absorbed into the fabric layer but some may lie upon the surface of the layer 172.

Referring to FIG. 18 another form 180 of the invention is shown in a cross section similar to FIG. 17, and in which corresponding features are numbered correspondingly. Visible symbols 181 have been printed onto the surface 171B using a conventional printing technique, for example ink jet printing. The above-mentioned wax is relatively easy to print on. On the upper surface 171B of the plastically deformable material 171, there is a cover layer 182, being the same wax material as the plastically deformable material 171, so that the symbols 181 are sandwiched between layers 171 and 182, in effect the symbols 181 being embedded in the plastically deformable material 171, 182. This cover layer 182 is translucent, and allows the visible symbols 181 to be seen through this layer 182. The cover layer 182 is applied to layer 171 by for example casting (e.g. slot die casting) the second wax layer 182 directly onto the first layer 171. Alternately the second layer 182 may be applied by pressing the strip 171 and a sheet of the material of layer 182 together, with heating, so that the strip 171 and layer 182 merge into a monolithic mass of the wax. The merged layers 171 and 182 can then be sized e.g. by pressing to a suitable thickness e.g. ca. 0.5 mm.

The layer of absorbent material 172 may then be applied to the layer 171 in a manner analogous to that described above for FIG. 17. The overall thickness of the three layers 182, 171, 172 is ca. 0.7 mm.

The pressing operations described above may be accomplished by passing the various strips through compressing rollers, in a manner conventional to the those skilled in the art.

In the devices specifically illustrated in FIGS. 17-18 the typical loading of the gel 173 is ca. 250-350 mg per strip.

Referring to FIGS. 19-24 various overall shapes of the preferred form of the device of the invention.

FIGS. 19, 20 and 21 show devices suitable for the upper teeth. The device 190 of FIG. 19 is generally rectangular, ca. 7.5 cm×1.75 cm, and has slightly convex curved long sides 190A and 190B, with rounded corners. The devices 200 and 210 of FIGS. 20 and 21 are generally rectangular, 200 being ca. 7.5 cm×1.75 cm, and 210 being ca. 6×2.3 cm, and have slightly concave curved long sides 200A, 200B, 210A, 210B. From the long side 200B, 210B of each of device 200, 210 projects a generally rectangular tab 201, 211. In the angles between the edges of the tab 211 and the long side 210B of device 210 are small notches 212.

FIGS. 22, 23, and 24 show devices suitable for the lower teeth. The devices 220, 230, 240 are all generally rectangular being ca. 7 cm×2 cm. The devices 220, 230 have a slightly convex curved lower side 220A, 230A, but a slightly concave curved upper edge 220B, 230B. The device 240 of FIG. 24 has a straight lower side 240A and a slightly concave upper side 240B.

A combination of the device 210 for the upper teeth and 230 for the lower teeth is preferred.

FIGS. 25, 26 and 27 show a device 210 as shown in FIG. 21 applied to the teeth.

Referring to FIG. 25, a device 210 as shown in FIG. 21 has been applied to the user's upper teeth in a manner as follows. FIG. 25A shows the front row of the user's upper teeth, being incisor teeth 251, canine teeth 252 and the first pre-molar teeth 253. The view in FIG. 25 is looking straight at the teeth from in front of the user. The device 210 has been applied to the front surface of the teeth 251-253, with its upper long side 210A overlapping the gumline 254. The opposite long side 210B extends beyond the crowns 255 (generally) of the teeth 251-253 distant from the gumline 254, with the tab 211 extending downwardly. As shown in FIG. 25B The device 210 has been folded or bent along a longitudinal fold line 256 so as to come into contact with the rear surfaces of the teeth 251-253. The length of the device 210 is such as to cover the ends of the canine teeth 252 of the user. The absorbent fabric 172, with its loading of the tooth whitening substance 173, is in contact with the surfaces of the teeth 251-233. A device 230 may be applied analogously to the front surface of the lower teeth, again comprising incisor teeth, canine teeth and pre-molar teeth. Analogously the device 230 also folds or bends at its fold line to come into contact with both the crowns of the teeth and the rear surfaces of the teeth.

Referring to FIG. 26, a plan view of the line of the user's teeth 251-253 is shown in FIG. 26A, extending from the incisor teeth 251 to the first pre-molars 253. The device 210 has been applied to the front surfaces of the teeth 251-253 and a firm pinching action has been applied to plastically deform the layer of wax 171. The absorbent fabric 172, with its loading of the tooth whitening substance 173, is in contact with the surfaces of the teeth 251-233. The pinching pressure has caused the device 210 to accommodate itself to the overall curve of the front surface of the teeth 251-253, and also to the concavities of the gaps 261 between the user's teeth 251-253. The device 260 has also been folded over the crowns of the teeth 251-253, so that the device 260 has also accommodated itself to the overall curve of the back surface of the teeth 251-253, and to the concavities of the gaps 261 between the user's teeth at the back surface.

FIG. 27 shows a cross section through an individual tooth 271 of the row 251-253 shown in FIG. 26 projecting from the gum 272, with the device 210 applied to its surface, with the fabric layer 172 and substance 173 in contact with the tooth 271. A firm pinching action applied by the user has caused the strip 210 to accommodate itself to the curves of the front teeth surface 271A and also to fold over the crown 273 of the tooth 271, and to contact the rear surface of the tooth 271B, accommodating itself to the curves of the rear surface 271B also.

This close fitting of the device 210 to the overall and individual shape of the teeth 251-253 achieved by the plastic deformation of the wax layer 171, 182 causes the device 210 to grip onto the user's teeth by friction and deadfold and to be retained in place during a suitable period of use. The device 210 may be retained in contact with the teeth 251-253 for a suitable period before being removed by the user, involving lifting the device 210 off the teeth, followed by rinsing the mouth if felt necessary.

FIG. 28 shows a cross section through a further device 280 of this invention. This is a multiple layered strip comprising an outer backing layer 281 and an inner contact layer 282 to be placed adjacent a tooth or other oral tissue surface. Both the outer and inner layers 281 282 are made of a plastically deformable dental wax. The inner layer is rendered porous by being perforated with numerous small holes 283. An oral healthcare substance 284 is provided in the pocket between the inner 281 and outer 282 layers, such that pressing the layers 281, 282 towards each other during use releases the substance 284 through the holes 284 during use. The outer 281 and inner 282 layers are laminated together at 285 around the perimeter of the pocket. The inner contact layer 282 is covered with a removeable protection layer 286 prior to use to prevent loss of substance 284 through the holes. The layer 286 can be peeled off in the direction shown by pulling tab 287.

FIG. 29 shows a cross section through a further device 290 of this invention. This comprises a strip 291 comprising a plastically deformable wax, on a surface of which are deposited microcapsules 292 containing an oral healthcare substance, stuck thereto using a thin film of adhesive (not shown). The capsules 292 are covered by a porous membrane layer 293 of a non-woven fabric material helping to retain the capsules 292 on the surface of the strip and to help to retain capsule casing debris when the capsules 292 have opened to release their content, whilst allowing active material content to pass through. The membrane layer 293 is bonded to the strip at 294 around the region in which the capsules 292 are deposited. In an alternative form the capsules 292 may be deposited on the surface of strip 291, and the membrane 293 may be adhered to the surface of the strip 291 using an adhesive.

FIG. 30 shows a cross section through a further device 300 of the invention, which comprises a layer 301 of a plastically deformable wax, of the same type as described above, ca. 0.7 mm thick. This extra thickness helps to give the wax layer strength in the absence of a non woven layer. On a surface of the strip 301 is deposited a layer 302 of a peroxide-containing gel. The device 300 can be made in shapes similar to those illustrated in FIGS. 19 and 20. For example such a device 300 may be provided without the layer 302 and a user may apply the layer 302 from a separate container. The device 300 can be adapted to the contours of the user's teeth and be attached thereto by friction deadfold etc. as above.

Devices 170-300 may be applied simultaneously to both the user's upper and lower teeth. It has been found that the wax strips 171 of the respective upper and lower strips do not generally stick together on contact whilst both upper and lower strips are worn. However only one device 170-290 may be used at a time, applied to only the upper or lower teeth.

FIGS. 31 and 32 show a sachet 310 suitable for a device 10, 30, 40, 50, 60, 70, 80, 90, 100, 110, 120, 130, 140, 150, 160, 170, 180, 190, 200, 210, 220, 230, 240, 280, 290 of this invention. The sachet 310 comprises a flat envelope defined by an envelope wall 311 of a foil laminate of a shape closely conforming to the shape of the device e.g. 10. A section 312 of the envelope wall 311 of the sachet 310 can be peeled away by pulling a tab 313, to form an opening in the sachet 310 by which the device e.g. 10 contained therein may be removed for use.

1. A delivery device for delivering an oral healthcare substance to the oral surfaces of the teeth, gingival and/or mucosal tissues, comprising; a strip of an orally acceptable flexible material, having a strip surface capable of being applied to a tooth surface and/or adjoining soft tissue, the strip having an oral healthcare substance deposited upon a strip surface thereof, and/or impregnated into its bulk, the substance being capable of transference from the strip surface to the tooth surface and/or adjoining soft tissue, the strip being capable of adhesion to a tooth surface and/or adjoining soft tissue to which the strip is applied, the adhesion function being provided independent of the oral healthcare substance.

2. A delivery device according to claim 1 wherein the strip comprises a plastically deformable material.

3. A device according to claim 2 wherein the plastically deformable material comprises a wax.

4. A device according to claim 2 wherein the plastically deformable material when in the form of the strip may be deformed by the user using finger or hand pressure to fit the device to the overall shape of the user's teeth.

5. A device according to claim 4 made of a plastically deformable material such that physical conformation of the plastically deformable material to the shape of the teeth causes attachment of the device to the teeth.

6. A device according to claim 4 wherein the material is capable of being plastically deformed so that the device can be folded or bent over the user's teeth from the front to the back surface of the teeth, so as to be in contact with both the front and back surfaces.

7. A delivery device according claim 1, wherein the strip comprises an inner carrier layer able to carry the substance and to be placed in contact with a user's teeth, and an outer barrier layer.

8. A delivery device according to claim 7 which comprises a barrier layer of a plastically deformable material, to at least one surface of which is attached a carrier layer of an absorbent material, an oral healthcare substance being on the carrier layer of absorbent material.

9. A device according to claim 8 wherein the absorbent material is an absorbent fabric.

10. A device according to claim 9 wherein the fabric is non-woven.

11. A device according to claim 10 wherein the non-woven fabric is a polypropylene-viscose blend.

12. A device according to claim 1, wherein the oral care substance and an adhesive material are deposited in separate discrete locations in relation to the strip surface.

13. A device according to claim 12 wherein the adhesive is deposited in places on the strip surface that enable part of the strip to stick to an oral surface adjacent to a tooth surface, so that another part of the strip on which the substance is deposited or into which it is impregnated contacts the tooth surface.

14. A device according to claim 12 wherein the oral healthcare substance and adhesive are deposited on the surface in respective discrete spots or patches or lines on the surface.

15. A device according to claim 14 wherein the oral healthcare substance is deposited in one or more patch bordered partly or completely surrounded by a border of the adhesive.

16. A device according to claim 11 wherein the adhesive and oral healthcare substance are deposited in separate discrete layers on the surface of the strip.

17. A device according to claim 1, wherein the strip material is inherently adherent to a tooth surface.

18. A device according to claim 1, wherein mechanical adhesive means provides an adhesive function, either alone or in combination with any other adhesive means.

19. A device according to claim 1, wherein the oral care substance and/or adhesive are encapsulated.

20. A device according to claim 1, wherein the oral care substance and/or adhesive are provided in granules which may release their content under the influence of the mouth environment.

21. A device according to claim 19 wherein said granules or capsules are covered by a porous membrane layer.

22. A device according to claim 1, comprising an outer backing layer and an inner contact layer to be placed adjacent a tooth or other oral tissue surface, the inner layer being porous and the oral healthcare substance being provided between the inner and outer layers.

23. A delivery device according to claim 1, marked with one or more visible symbol.

24. A delivery device according to claim 23 wherein a cover layer is applied over the one or more symbol.

25. A device according to claim 23 which comprises a barrier layer of a plastically deformable material, to at least one surface of which is attached a carrier layer of an absorbent material, an oral healthcare substance on the carrier layer of absorbent material, and marked with one or more visible symbol applied to the surface of the plastically deformable material opposite to the surface on which is attached the layer of an absorbent material, and having a cover layer over the one or more symbol.

26. A device according to claim 25 wherein the cover layer is the same material as the plastically deformable material so that the symbol is in effect embedded in the plastically deformable material.

27. A delivery device according to claim 1, wherein the oral care substance is a tooth whitening substance.

28. A delivery device according to claim 1, wherein the oral care substance is in the form of a gel.

29. A delivery device according to claim 27 wherein the oral healthcare substance is a peroxide-containing gel.

30. A device according to claim 1, being of elongate shape of a length sufficient that when placed against the front surface of the teeth of a user it extends across a plurality of teeth, and of sufficient width that when placed against the user's teeth it extends from the gumline at least to the crowns of the front teeth distant from the gumline.

31. A device according to claim 30 wherein the width is such that in an unfolded state the strip has an unfolded width greater than the height of the teeth from the gumline to the crown, and at least part of the strip may be folded about a substantially longitudinal fold axis so as to bend or fold over the crowns and contact the crowns and rear surfaces of the user's teeth.

32. A device according to claim 30 being substantially rectangular with concavely curved long sides.

33. A device according to claim 30 substantially rectangular with convexly bowed long sides, or of a generally rectangular shape but with a concave curved long side or a concave indentation in a long side.

34. A device according to claim 1 having a tab extending from a long side.

35. A device according to claim 34 wherein at least part of the strip may be folded into a folded cross section substantially of a “U” or “V” shape, with two limbs linked at a fold axis each limb with an inward surface facing into the “bite” of the “U” or “V”, with the oral healthcare substance present on this inward, tooth-contacting surface.

36. A method of treating teeth with a device according to claim 1 in which the device is applied manually to a user's tooth surface(s), with the length dimension of the strip aligned with the line of the user's teeth, the substance-bearing surface in contact with the front surface of the teeth, and the device is pressed against the front surface of the teeth, leaving the device in contact with the user's teeth for a sufficient length of time for the oral healthcare substance to have its effect, then removing the device.

37. A process for making a device according to claim 8 comprising bringing the absorbent material into contact with the surface of the strip and applying pressure, then applying the oral healthcare substance to the absorbent material.

38. A process according to claim 37 comprising;
providing a dental wax in the form of a sheet,
optionally cleaning the surface of the sheet of the wax material,
printing a visible symbol upon a first surface of a first sheet of the wax material,
laminating a second, cover sheet of the wax material to the first surface of the first sheet, to sandwich the printed symbol between the first and second sheets,
sizing the laminated sheets by compressing them to a suitable thickness,
attaching the layer of absorbent fabric to the laminated wax strip,
cutting the sheet of laminated material to shape,
applying the oral healthcare substance to the layer of absorbent material.

39. A device according to claim 3-wherein the plastically deformable material when in the form of the strip may be deformed by the user using finger or hand pressure to fit the device to the overall shape of the user's teeth.

40. A device according to claim 5 wherein the material is capable of being plastically deformed so that the device can be folded or bent over the user's teeth from the front to the back surface of the teeth, so as to be in contact with both the front and back surfaces.

41. A delivery device according to claim 6 wherein the strip comprises an inner carrier layer able to carry the substance and to be placed in contact with a user's teeth, and an outer barrier layer.

42. A device according to claim 11 wherein the oral care substance and an adhesive material are deposited in separate discrete locations in relation to the strip surface.

43. A device according to claim 13 wherein the oral healthcare substance and adhesive are deposited on the surface in respective discrete spots or patches or lines on the surface.

44. A device according to claim 20 wherein said granules or capsules are covered by a porous membrane layer.

45. A device according to claim 24 which comprises a barrier layer of a plastically deformable material, to at least one surface of which is attached a carrier layer of an absorbent material, an oral healthcare substance on the carrier layer of absorbent material, and marked with one or more visible symbol applied to the surface of the plastically deformable material opposite to the surface on which is attached the layer of an absorbent material, and having a cover layer over the one or more symbol.

46. A delivery device according to claim 28 wherein the oral healthcare substance is a peroxide-containing gel.

47. A device according to claim 31 being substantially rectangular with concavely curved long sides.

48. A device according to claim 1 substantially rectangular with convexly bowed long sides, or of a generally rectangular shape but with a concave curved long side or a concave indentation in a long side.

49. A device according to claim 33 having a tab extending from a long side.
